(19) United States
(12) Patent Application Publication
(10) Pub. No.: US 2007/0166700 A1
(43) Pub. Date: Jul. 19, 2007

(54) Sensing biological analytes on a ferroelectric transducer

(76) Inventors: Ooi Kiang Tan, Singapore (SG); Wei Qiu, Singapore (SG); Yik Yuen Gan, Singapore (SG); Xi Yao, Shanghai (CN); Swee Chuan Tjin, Singapore (SG)

Correspondence Address: JACOBSON HOLMAN PLLC, 400 SEVENTH STREET N.W., SUITE 600, WASHINGTON, DC, 20004, US

(21) Appl. No.: 10/587,542

(22) PCT Filed: Jan. 28, 2005
(86) PCT No.: PCT/SG05/00024

§ 371 Date: Jul. 28, 2006

Related U.S. Application Data
(60) Provisional application No. 60/540,069, filed on Jan. 30, 2004.

(51) Int. Cl. C12Q 1/70; C12Q 1/68; G01N 33/53; G01N 33/569; G01N 33/554; C12M 3/00

(52) U.S. Cl. 435/5; 435/6; 435/7.1; 435/7.31; 435/7.32; 435/287.2; 205/777

(57) ABSTRACT

A method of detecting a biological analyte within a sample (12) is provided. The analyte is one that can be electrically charged or polarized in the presence of an electric field. The sample is place in proximity with a ferroelectric transducer (14). An electric field is established to polarize the analyte in the sample. An electric response of the ferroelectric transducer resulting from the electric field and indicative of the presence of the analyte in the sample is then sensed. Also provided is a sensor for detecting the analyte within the sample. The sensor has a ferroelectric transducer and first (20) and second electrodes (22) for establishing a potential difference across a sample disposed adjacent to the transducer to generate an electric field in the sample. The sensor may also have an electric signal detector (26) for sensing the electric response.

### CROSS-REFERENCE TO RELATED APPLICATION

This application claims priority from U.S. provisional application no. 60/540,069, entitled “FERROELECTRIC FILMS FOR BIOLOGICAL SENSING AND DETECTION APPLICATIONS” and filed Jan. 30, 2004, which is incorporated herein by reference.

### FIELD OF THE INVENTION

The present invention relates generally to sensing analytes, and more particularly to method and apparatus for sensing biological analytes.

### BACKGROUND OF THE INVENTION

Biosensors are sensors for sensing biological analytes. Biosensors have wide-spread applications in various fields such as medicine, environmental protection, food processing, security, defence, and the like.

Known biosensors can be classified based on their transduction methods, which include three main types—optical transduction, electrochemical transduction, and piezoelectric transduction. However, each of these three types of biosensors has some shortcomings.

For example, optical biosensors may require delicate and expensive instrumentation. Low signal to noise ratios can result from ambient light. The dynamic range of detection can be small in comparison with electrical sensors. Further, signal intensity is dependent on sample volume and thus it may be difficult to detect a small volume of sample.

The electrochemical biosensors typically have low sensitivity.

The piezoelectric transducers in piezoelectric biosensors can be fragile which limits their application.

Thus, there is a need for a biosensor or a transducer for biosensors that is relatively simple in structure, easy and inexpensive to manufacture, and/or highly sensitive. There is also a need for biosensors which has a disposable transducer.

### SUMMARY OF THE INVENTION

In one aspect of the present invention, there is provided a method of detecting a biological analyte within a sample. The analyte can be electrically charged or polarized in the presence of an electric field. The sample is placed in proximity with a ferroelectric transducer. An electric field is established to polarize the analyte in the sample. An electric response of the ferroelectric transducer resulting from the electric field and indicative of the presence of the analyte in the sample is sensed.

In another aspect of the invention, there is provided a sensor for detecting a biological analyte within a sample, wherein the analyte can be electrically charged or polarized in an electric field. The sensor comprises a ferroelectric transducer; a biological sample disposed adjacent the transducer; first and second electrodes for establishing a potential difference across the sample to generate an electric field in the sample; and an electric signal detector for sensing an electric response of the ferroelectric transducer resulting from polarization of the analyte, and indicative of the presence of the analyte in the sample.

Other aspects, features, and benefits of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate exemplary embodiments of the invention,

FIG. 1 is a schematic diagram of a biosensor;

FIG. 2 is a cross-sectional view of a ferroelectric transducer;

FIG. 3 is a schematic cross-sectional view of analytes immobilized on a transducer;

FIG. 4 is a line graph of detected voltage/current versus concentration for a sample; and

FIGS. 5 to 10 are line graphs of voltage shift versus concentrations for several biological samples.

### DETAILED DESCRIPTION

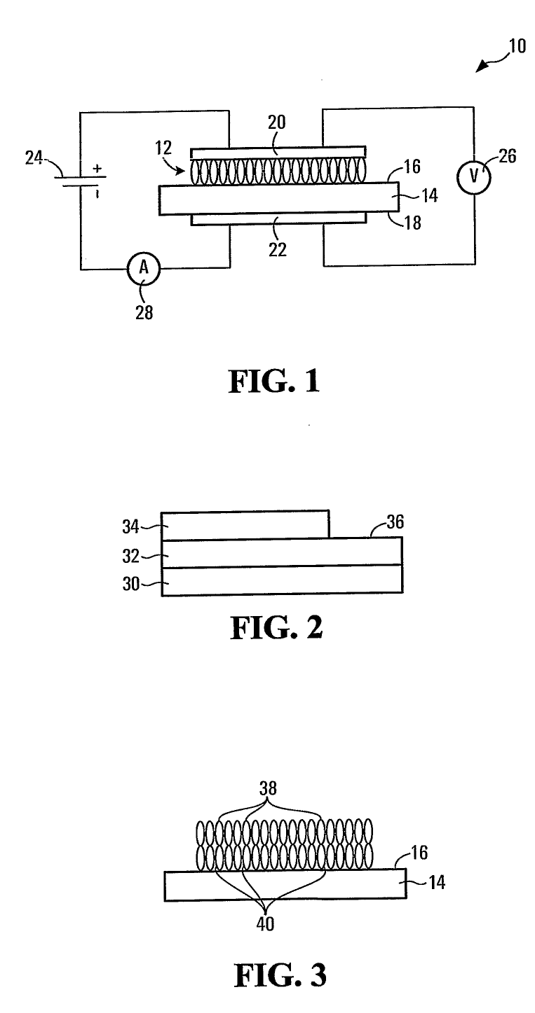

FIG. 1 is a schematic diagram of a biosensor 10 for detecting a biological analyte within a sample, exemplary of an embodiment of the present invention. A biosensor is a sensor suitable for detecting or sensing biological analytes. Biological analytes include proteins, DNAs, viruses, antigen-antibody, bacteria, fungus, drugs, and the like. Biosensor 10 is suitable for detecting biological analytes which can be electrically polarized or charged in the presence of an electric field. A biological sample, or biosample such as biosample 12, is a sample that potentially includes one or more biological analytes.

Biosensor 10 includes a ferroelectric transducer 14. Transducer 14 may be generally plate-shaped or film-shaped and has a top surface 16 and a bottom surface 18. The top surface 16 contacts biosample 12. Biosensor 10 also includes two electrodes 20 and 22 for establishing a potential difference across biosample 12 and transducer 14. Top electrode 20 is in contact with biosample 12 and bottom electrode 22 is in contact with bottom surface 18 of transducer 14. Electrodes 20 and 22 are connected to source 24 that applies a voltage across electrodes 20 and 22 and hence biosample 12 and transducer 14. In the depicted embodiment, electrodes 20 and 22 are flat plates.

An electric signal detector such as voltmeter 26 or ammeter 28 is operably connected or positioned to detect an electric signal from the electrodes 20 and 22 when the voltage is applied.

Transducer 14 is formed, at least in part, of a ferroelectric material such as BaₓSr₁₋ₓTiO₃ (BST) or Pb(ZrₓTi₁₋ₓ)O₃ (PZT), where “x” can be any number between 0 and 1. As will be appreciated, BST can become non-ferroelectric at temperatures above its Curie temperature, which is dependent on the value of “x”. The ferroelectric material may also be a ferroelectric polymer, which may or may not be doped with a doping element such as lanthanum or manganese. The ferroelectric material can be in an amorphous, polycrystalline, or nano-structured phase. The ferroelectric material may have any suitable shape and size. For example, it can form a thin film with a suitable thickness. Typical thickness can vary between about 160-200 nm, but can be up to, for example, 1 μm. A thicker film can allow a higher voltage thus increasing the sensitivity of the sensor, but may be more expensive and difficult to fabricate. Thus, it may not be economically desirable to have a ferroelectric layer thicker than necessary. A BST film about 180 nm thick has been found adequate for detecting certain biological analytes. Top surface 16 of transducer 14 may or may not be formed with a ferroelectric material. Top surface 16 may be formed or treated with a material suitable for immobilizing biosample 12 thereon. For example, top surface 16 may have a coating material to which target analytes can directly attach. Top surface 16 may also be coated with molecules that have specific affinity to the target analytes (referred to as “probe molecules”). Because probe molecules have specific affinity to the target analytes, they will selectively capture or bind the target analytes. Suitable probe molecules will depend on the target analyte and ferroelectric material used, as will be understood by persons skilled in the art.

Electrodes 20 and 22 can be any suitable electrodes. Similarly, source 24 and signal detectors 26 and 28 can be any suitable source or detectors. Suitable electrodes, signal sources and detectors will be known to persons skilled in the art. For example, a multimeter or an oscilloscope may be used to measure both voltage and current from electrodes 20 and 22. The signal source can be a direct current (DC) or alternative current (AC) source and may provide a constant voltage or current. As can be understood, source 24 and detectors 26 and 28 can be integrated. Top electrode 22 may be spaced from transducer 14 at a fixed distance, or may be moveable relative to transducer 14.

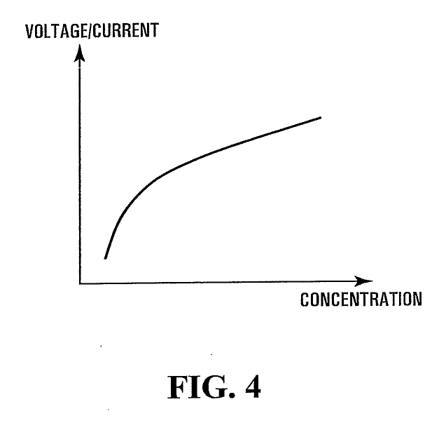

Transducer 14 and bottom electrode 22 can be formed on a silicon wafer using known semiconductor techniques. An exemplary procedure for forming a BST transducer on a silicon wafer is described with reference to FIG. 2 for illustration purposes. As illustrated, a silicon wafer 30 is coated with a platinum layer 32. A BST film 34 is formed on platinum layer 32. As can be appreciated, platinum layer 32 is the bottom electrode and film 34 is the transducer. Film 34 can be formed by spin-coating a BST sol solution onto layer 32. The BST sol solution can be prepared in any suitable manner, which can be readily determined or understood by a person skilled in the art.

For example, BST sol solution can be formed by mixing commercially available titanium butoxide (Ti(OC₄H₉)₄), barium acetate (Ba(CH₃COO)₂) and strontium acetate (Sr(CH₃COO)₂) to form a precursor solution, and adding acetylacetone (C₅H₇OOH) and 2-ethoxy-ethanol (C₄H₉OOH) to stabilize the solution. The BST sol solution can be spun onto the cleaned platinum layer 32 at 4000 rpm for 30 seconds. Multiple layers of BST can be coated to obtain a desired film thickness. For example, four layers of spin-coated BST can form a total thickness of about 180 nm. After final coating, the ensemble can be annealed, for example, at 475° C. in air for about one hour in a quartz furnace. In order to make electric connection to bottom electrode 32, a portion of the BST film can be etched off, for example by using 1:5 di-ionized water diluted HF solution, so as to expose a portion such as portion 36 of bottom electrode 32.

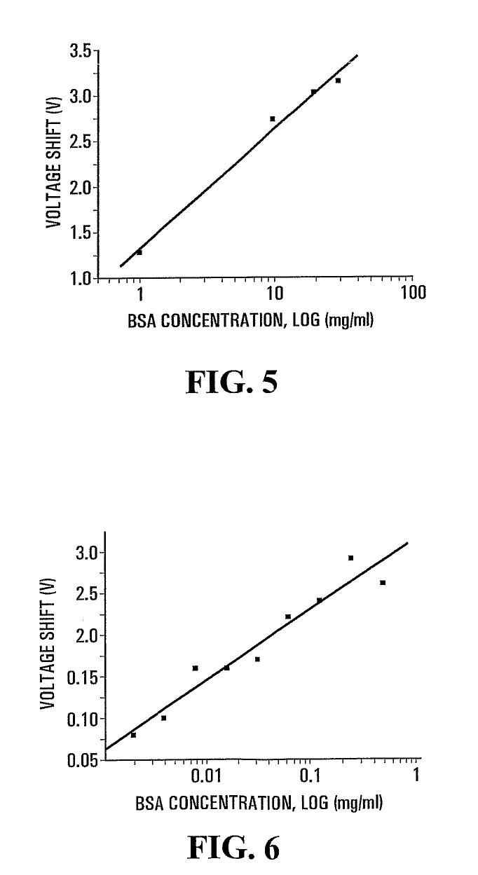

Biosample 12 may be a liquid and can be introduced onto top surface 16 either by direct liquid dropping, such as by using a pipette (not shown), or through a fluid channel (not shown). The fluid channel may be small in cross-section which can be in the micrometer scale. Sensor 10 may be housed in a chamber which is in fluid communication with the fluid channel. For example, such a chamber and channel may be formed on a micro-chip. The biological analytes to be detected should be electrically polarisable or become charged under an electric field. The analytes in biosample 12 may be mobile or immobilized on top surface 16 (FIG. 1) of transducer 14. For example, analytes may be immobilized directly on top surface 16. Alternatively, as shown in FIG. 3, the analytes in biosample 12, such as analyte 38, may be “captured” by or bond to probe molecules, such as probe molecules 40 which are attached to the top surface 16 of transducer 14.

The signal detector can detect one or more of voltage, current, electrical charge, resistance, capacitance or other electrical properties that can be different between signals obtained for biosample 12 and a reference sample. The signal detector can also include a circuit for signal amplification, noise deduction or other purposes.

As can be understood, biosensor 10 can be a capacitive, resistive, diode or transistor type sensor. For example, biosensor 10 and biosample 12 together can form two parallel capacitors.

In operation, source 24 establishes a potential difference (voltage) across electrodes 20 and 22 and hence biosample 12. The potential difference may have a pre-selected voltage value or be adjusted to maintain a pre-selected current flow through electrodes 20 and 22. The pre-selected voltage or current may vary depending on a number of factors such as the intended application, the transducer material and thickness, the analyte or sample type, distance between the electrodes, and the like. Typically, the voltage may be in the range of about 1 to 100 volts, and the current may be on the order of nA or μA.

For ease of description, it is assumed below that a constant current (I) is maintained across electrodes 20 and 22. The pre-selected current level can be maintained by monitoring the current through ammeter 28 and adjusting the output of source 24, either manually or automatically. In any event, the potential difference establishes an electric field within biosample 12. This field, in turn, polarizes or charges analyte (or a fraction thereof) within the biosample 12.

Biosample 12 is in proximity with transducer 14. The target analyte in biosample 12 may be immobilized on top surface 16 of transducer 14, either directly attaching to top surface 16 or by binding to probe molecules, such as probe molecules 40, attached to top surface 16. When an immobilization step is performed, the remaining portion of biosample 12 may be removed after immobilization, such as by washing.

As will be appreciated, the permanent electric dipole moment possessed by the ferroelectric material of transducer 14 may be reoriented by the application of an electric field. The effect of this field on transducer 14, in turn affects the current/voltage across transducer 14.

A response signal, in this example case the voltage (V_{S}) across electrodes 20 and 22, is detected using the signal detector, in this case voltmeter 26. The response signal is indicative of the effect of the electric field in biosample 12 on transducer 14. This voltage is compared with a reference voltage V_{R}, which is the response voltage that would have been detected if biosample 12 were replaced with a reference sample while other conditions were substantially the same. The reference sample can be a blank sample or a sample of the sample type as biosample 12. A blank sample is one that does not contain any target analytes. It may be advantageous if the blank sample is not electrically charged and has no or little electric polarization in an electric field. The reference sample can be a buffer solution such as de-ionized water. The reference voltage (V_{R}) can be measured simultaneously or sequentially with the sample voltage (V_{S}), using the same biosensor or separate biosensors. The reference voltage V_{R} may also be obtained from a previously conducted measurement, or from a database or a standard reference.

As can be appreciated, it is possible to determine the concentration of the target analyte in biosample 12 if the analyte is of a known type. The concentration can be indicated by the difference between the sample response signal and the reference signal, which will be referred to herein as a signal shift, such as a voltage shift ΔV=V_{S}−V_{R}, when the current (I) is maintained constant. Similarly, when the voltage is the same for both biosample 12 and the reference sample, a current shift (ΔI) may result and the response signal can be the current and it is possible to detect analytes by establishing a constant voltage and detecting the current shifts.

The signal shift may also be used to indicate the presence of different types of analytes as they may produce very different signal shifts.

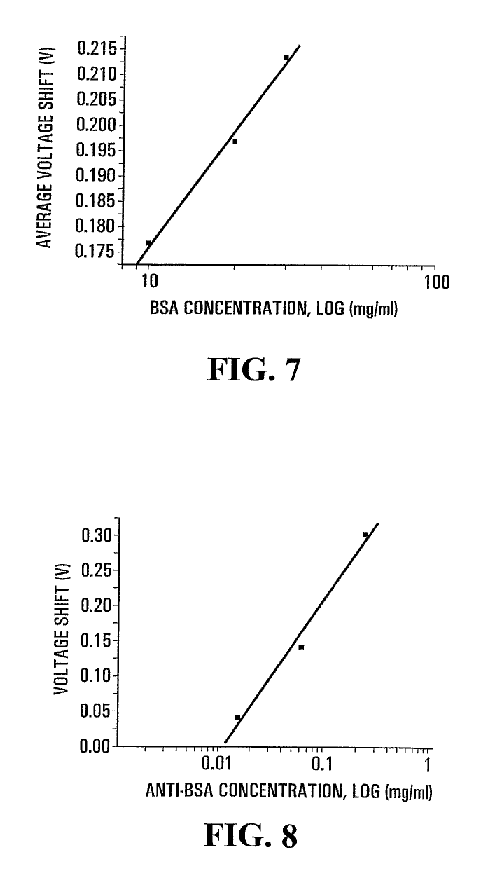

FIG. 4 shows an example line graph of voltage/current signal shift versus concentration of analyte within biosample 12. Thus, reference voltages for a range of concentrations of the same type of analyte as the target analyte in biosample 12 can be obtained and tabulated or plotted, and the concentration in biosample 12 can be determined by matching measured V_{S} to table or plot. It is closest to the concentration corresponding to the V_{R} that matches V_{S} most closely in some situations, it may be necessary to measure a number of V_{S} at different conditions (e.g. different current levels) to determine the type of biosample 12.

It should be noted that other factors, such as temperature and the amount of biosample 12 or the analytes immobilized on sensor 10, may also affect the signal shift. Thus, these conditions may need to be taken into account when comparing sample signal shifts.

As should now be understood, while it may be possible to directly observe signal shifts between a biological sample and a reference sample without using a ferroelectric transducer, the presence of a ferroelectric transducer can enhance the signal shifts or make them easier to detect. Without being limited to a particular theory, one possible explanation for the enhancement is that a ferroelectric transducer can have a high dielectric constant and thus a high electric potential difference can be induced across the transducer when it is placed adjacent an electrically polarized sample. The polarized sample creates an external field in transducer 14 which polarizes transducer 14. When biosample 12 contains analytes that are electrically polarized or charged under a potential bias, biosample 12 becomes electrically polarized. Usually, the higher the concentration of the analyte, the higher the polarization. Thus, the resulting signal shift can be more pronounced when a ferroelectric transducer and biosample 12 are placed adjacent to each other as compared to using no transducer or a non-ferroelectric transducer.

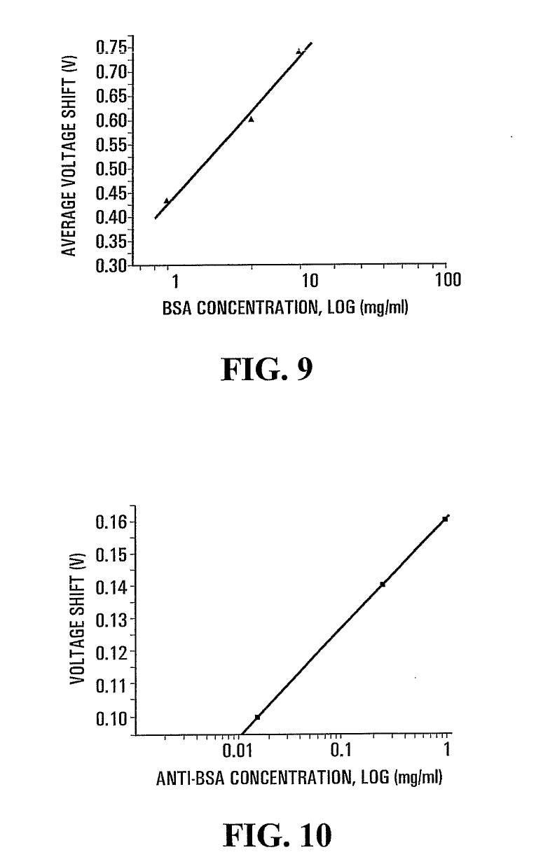

To further illustrate, example relationships between voltage shifts and sample concentrations are shown in FIGS. 5 to 10. Reference signals were obtained with a buffer solution containing de-ionized water. As can be seen in each figure, the voltage shifts are linearly dependent on the logarithm values of the concentrations of the analytes.

FIGS. 5 and 6 show the results obtained with a Bovine Serum Albumin (BSA) solution as the biosample and a BST film as the transducer. For each measurement, 10 μl of BSA solution was dropped onto the BST film. A direct voltage was applied to top and bottom electrodes and was increased from 1 to 10 V with incremental of 20 mV. The voltage shifts were measured for different concentrations of BSA at a leakage current of 6 μA in FIG. 5 and 0.4 μA in FIG. 6, respectively. The BSA concentrations were 1, 10, 20 or 30 mg/ml respectively for the data points shown in FIG. 5 and from 1/512 to ½ mg/ml for FIG. 6.

FIG. 7 shows the results obtained with immobilized BSA samples. The transducer used included a BST thin film. To immobilize the BSA sample, a thin layer of Au (about 200 nm thick) was coated on top of the BST thin film using an evaporation-beam method. The Au surface was cleaned by sonication for one hour in ethanol solution. 50 mg ProLinker™ B was dissolved in 60 ml of chloroform (CHCI₃) with a final concentration of 1 mM. The Au surface was immersed in the ProLinker™ B—CHCI₃ solution for one hour. As a result, a monolayer (SAM) of ProLinker™ B was formed on the Au surface through a self-assembling process. The final surface was rinsed with CHCI₃, acetone, de-ionized water, ethanol, and then dried in a pure N₂ stream. The final surface was then immersed in a phosphate buffered saline (PBS) solution for one hour at room temperature, which had BSA concentrations of 10, 20 and 30 mg/mL respectively. Again, a direct voltage was applied to the top and bottom electrodes when about 10 μl di-ionized water was dropped onto the immobilized BSA surface. The leakage current was 6 μA.

FIG. 8 shows the results for anti-BSA samples which were bound to immobilized BSA as described above where the concentration of the BSA was 10 mg/ml. For each measurement, 10 μl anti-BSA was dropped onto the immobilized BSA and was allowed to stay overnight. The sample surface was rinsed with di-ionized water and then dried by N₂. A direct voltage was applied to the electrodes and the leakage current was 6 μA. The anti-BSA concentrations were 1/64, 1/16 and ¼ mg/ml respectively. The sensitivity for detection of anti-BSA concentration can be improved, for example, by choosing an optimized immobilized BSA concentration or a larger leakage current.

FIG. 9 shows the results obtained with BSA samples being immobilized by covalent bonding with dodecyl phosphate (DDPO₄). In this case, the BSA was immobilized directly on a BST film. The BST surface was cleaned using O₂ plasma for three minutes and immersed in DDPO₄ solution for 48 hours. The surface was rinsed with di-ionized water and dried with pure N₂. The BST surface was then immersed in a PBS solution containing BSA for one hour at room temperature, wherein for different measurements the solution had different BSA concentrations at 10, 20 and 30 mg/ml respectively. 10 μl di-ionized water was dropped onto the immobilized BSA surface and a direct voltage was applied. The leakage current was 30 μA. Compared with the results shown in FIG. 8, the detection sensitivity in this case has been improved, perhaps due to the larger leakage current.

FIG. 10 shows the results obtained from anti-BSA samples which were bound to BSA immobilized on the BST film as described above. The leakage current was 6 μA. The selected concentration of immobilized BSA was 20 mg/ml. The anti-BSA concentrations were 1/64,¼, and 1 mg/ml.

As now can be appreciated, biosensor 10 can be used to determine the types and concentrations of biological analytes in samples and can have some advantages over conventional biosensors. For example, it can have a simple structure, can be inexpensive, and can have high sensitivity and fast response time. Since transducer 14 can be formed using known techniques on a silicon wafer, biosensor 10 can be produced using currently available semiconductor techniques, which are mature and suitable for mass-production.

Although only exemplary embodiments of this invention have been described above, those skilled in the art will readily appreciate that many modifications are possible therein without materially departing from the novel teachings and advantages of this invention. The invention, rather, is intended to encompass all such modification within its scope, as defined by the claims.

1. A method of detecting a biological analyte within a sample, wherein said analyte can be electrically charged or polarized in the presence of an electric field, said method comprising:
placing said sample in proximity with a ferroelectric transducer;
establishing an electric field to polarize said analyte in said sample;
sensing an electric response of said ferroelectric transducer resulting from said electric field and indicative of the presence of said analyte in said sample.

2. The method of claim 1, further comprising determining a signal difference between said electric response and a reference signal, said signal difference indicative of the presence of said analyte.

3. The method of claim 2, wherein said signal difference is indicative of the concentration or density of said analyte.

4. The method of claim 1, comprising disposing said transducer and said sample between first and second electrodes, and applying a voltage to said first and second electrodes to establish said electric field in said sample.

5. The method of claim 4 wherein said first electrode is in contact with said transducer and a second electrode is in contact with said sample.

6. The method of claim 4 wherein said electric response is said voltage when a pre-selected electric current is flowing between said electrodes.

7. The method of claim 4 wherein said electric response is the electric current flowing through said electrodes when said voltage has a pre-selected value.

8. The method of claim 1, wherein said ferroelectric transducer comprises one or more of BaₓSr₁₋ₓTiO₃ (BST), Pb(ZrₓTi₁₋ₓ)O₃ (PZT) and ferroelectric polymers, wherein x is between 0 and 1.

9. The method of claim 1 wherein said transducer is a thin film.

10. The method of claim 1 wherein said analyte is one of protein, DNA, virus, antigen-antibody, bacteria, fungus, and drug.

11. The method of claim 1, wherein said placing comprises immobilizing said analyte in said sample on said transducer.

12. The method of claim 11, wherein said analyte is immobilized directly on a ferroelectric layer of said transducer.

13. The method of claim 11, wherein said immobilizing comprises binding said analyte to a probe molecule attached to said transducer, said probe molecule having specific affinity to said analyte.

14. The method of claim 12, further comprising, after immobilizing said analyte on said transducer and before said sensing, removing a remaining portion of said sample and attaching a probe molecule to said analyte, said probe molecule having specific affinity to said analyte, and wherein said electric response is indicative of the presence of said probe molecule and thus said analyte.

15. A sensor for detecting a biological analyte within a sample, wherein said analyte can be electrically charged or polarized in an electric field, said sensor comprising:
a ferroelectric transducer;
a biological sample disposed adjacent said transducer;
first and second electrodes for establishing a potential difference across said sample to generate an electric field in said sample; and
an electric signal detector for sensing an electric response of said ferroelectric transducer resulting from polarization of said analyte, and indicative of the presence of said analyte in said sample.

16. The sensor of claim 15 further comprising a source connected to one or more of said first and second electrodes for applying a voltage to said first and second electrodes.

17. The sensor of claim 15 wherein said ferroelectric transducer comprises one or more of BaₓSr₁₋ₓTiO₃ (BST), Pb(ZrₓTi₁₋ₓ)O₃ (PZT) and ferroelectric polymers, wherein x is between 0 and 1.

18. The sensor of claim 15 wherein said transducer is a thin film.

19. The sensor of any one of claim 15 wherein said analyte is one of protein, DNA, virus, antigen-antibody, bacteria, fungus, and drug.

20. The sensor of any one of claim 15, wherein said first electrode is in contact with said transducer and said second electrode is in contact with said sample.

21. The sensor of any claim 15 wherein said transducer is in contact with said sample.

22. The sensor of claim 15 wherein said analyte in said sample is immobilized on said transducer.

23. The sensor of claim 22, wherein said analyte is directly attached to said transducer.

24. The sensor of claim 22, further comprising a probe molecule attached to said transducer, said probe molecule having specific affinity to said analyte, said analyte being bond to said probe molecule.
